(19) United States
(12) Patent Application Publication
(10) Pub. No.: US 2022/0249154 A1
(43) Pub. Date: Aug. 11, 2022

(54) ELECTROSURGICAL SYSTEMS AND METHODS FOR SEALING TISSUE

(71) Applicant: Covidien LP, Mansfield, MA (US)

(72) Inventors: Allison C. Weghorst, Broomfield, CO (US); Jennifer R. McHenry, Denver, CO (US)

(21) Appl. No.: 17/530,621

(22) Filed: Nov. 19, 2021

Related U.S. Application Data
(60) Provisional application No. 63/148,418, filed on Feb. 11, 2021.

(51) Int. Cl. A61B 18/14; A61B 18/12

(52) U.S. Cl. CPC A61B 18/1445; A61B 2018/0063; A61B 18/1206

(57) ABSTRACT

A method of sealing tissue in accordance with the present disclosure include grasping tissue between first and second jaw members, applying energy to the grasped tissue in accordance with a pre-treatment algorithm to pre-treat the grasped tissue in anticipation of tissue sealing, and applying energy to the pre-treated, grasped tissue in accordance with a tissue sealing algorithm to seal the pre-treated grasped tissue. Electrosurgical systems configured to implement the method are also provided.

### CROSS-REFERENCE TO RELATED APPLICATIONS

This application claims the benefit of, and priority to, U.S. Provisional Patent Application No. 63/148,418, filed on Feb. 11, 2021, the entire contents of which are hereby incorporated herein by reference.

### FIELD

The present disclosure relates to electrosurgery and, more particularly, to electrosurgical systems and methods for sealing tissue.

### BACKGROUND

In bipolar electrosurgery, electrical current is conducted through tissue positioned between electrodes of different polarity to heat and thereby treat the tissue. Bipolar electrosurgery often involves the use of an electrosurgical forceps, a pliers-like instrument that relies on mechanical action between its jaws to grasp, clamp, and constrict tissue. Electrosurgical forceps, more specifically, utilize mechanical clamping action and electrical energy to treat, e.g., cauterize, coagulate, and/or seal, clamped tissue.

Whereas cauterization involves the use of heat to destroy tissue and coagulation is a process of desiccating tissue such that the tissue cells are ruptured and dried, tissue sealing is a process of liquefying the collagen, elastin, and ground substances in the tissue so that they reform into a fused mass with significantly reduced demarcation between opposing tissue structures. In order to create an effective tissue seal, two predominant mechanical parameters must be accurately controlled: the pressure applied to the tissue and the gap distance between the electrodes. In addition, electrosurgical energy must be applied to the tissue under controlled conditions, e.g., controlling the intensity, frequency, and duration of electrosurgical energy application to tissue, to ensure creation of an effective tissue seal.

### SUMMARY

As used herein, the term “distal” refers to the portion that is being described which is farther from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. Further, to the extent consistent, any or all of the aspects detailed herein may be used in conjunction with any or all of the other aspects detailed herein.

A method of sealing tissue in accordance with the present disclosure includes grasping tissue between first and second jaw members, applying energy to the grasped tissue in accordance with a pre-treatment algorithm to pre-treat the grasped tissue in anticipation of tissue sealing, and applying energy to the pre-treated, grasped tissue in accordance with a tissue sealing algorithm to seal the pre-treated grasped tissue.

In an aspect of the present disclosure, the tissue sealing algorithm is independent of the pre-treatment algorithm.

In another aspect of the present disclosure, the pre-treatment algorithm is controlled in a first manner and the tissue sealing algorithm is controlled in a second, different manner. At least a portion of the pre-treatment algorithm may be voltage-controlled. At least a portion of the pre-treatment algorithm may follow a voltage versus time graph including, in an aspect, a fixed positive voltage ramp. At least a portion of the tissue sealing algorithm may adjust energy output to track an impedance versus time trajectory.

In yet another aspect of the present disclosure, the method further includes determining whether the pre-treatment algorithm is complete before applying the energy to the pre-treated, grasped tissue in accordance with the tissue sealing algorithm.

In still another aspect of the present disclosure, determining whether the pre-treatment algorithm is complete includes comparing an overall impedance change to an impedance change threshold.

In still yet another aspect of the present disclosure, the method further include, after applying the energy to the grasped tissue in accordance with the pre-treatment algorithm and before applying the energy to the pre-treated, grasped tissue in accordance with the tissue sealing algorithm, implementing a delay period where no energy is applied.

Another method of sealing tissue in accordance with aspects of the present disclosure includes grasping tissue between first and second jaw members and determining whether pre-treatment of the grasped tissue is to be performed. In a case where it is determined that pre-treatment of the grasped tissue is to be performed, the method includes applying energy to the grasped tissue in accordance with a pre-treatment algorithm to pre-treat the grasped tissue in anticipation of tissue sealing, and applying energy to the pre-treated, grasped tissue in accordance with a tissue sealing algorithm to seal the pre-treated grasped tissue. In a case where it is not determined that pre-treatment of the grasped tissue is to be performed, the method includes applying energy to the grasped tissue in accordance with the tissue sealing algorithm to seal the grasped tissue.

In an aspect of the present disclosure, the tissue sealing algorithm is independent of the pre-treatment algorithm.

In another aspect of the present disclosure, determining whether pre-treatment of the grasped tissue is to be performed is based on sensor feedback, a determined size of the grasped tissue, a manual input, a clamping force applied by the first and second jaw members to the grasped tissue, and/or a gap distance between opposing surfaces of the first and second jaw members with the grasped tissue therebetween.

In yet another aspect of the present disclosure, in the case where it is determined that pre-treatment of the grasped tissue is to be performed, the method further includes determining whether the pre-treatment algorithm is complete before applying the energy to the pre-treated, grasped tissue in accordance with the tissue sealing algorithm.

In still another aspect of the present disclosure, determining whether the pre-treatment algorithm is complete includes determining an overall impedance change during the pre-treatment, determining a size of the grasped tissue during the pre-treatment, determining a clamping force applied by the first and second jaw members to the grasped tissue during the pre-treatment, and/or or determining a gap distance between opposing surfaces of the first and second jaw members during the pre-treatment.

In still yet another aspect of the present disclosure, at least a portion of the pre-treatment algorithm is voltage-controlled and/or at least a portion of the tissue sealing algorithm adjusts energy output to track an impedance versus time trajectory.

Electrosurgical systems, e.g., including an electrosurgical generator and an electrosurgical forceps, for implementing the above methods are also provided in accordance with the present disclosure.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of an electrosurgical system in accordance with the present disclosure including an electrosurgical forceps and an electrosurgical generator;

FIGS. 2A and 2B are enlarged, perspective views of a distal end portion of the forceps of FIG. 1 with an end effector assembly thereof disposed in spaced-apart and approximated positions, respectively;

FIG. 3 is a schematic illustration of a robotic surgical system configured for use in accordance with the present disclosure;

FIG. 4 is a block diagram of the generator of FIG. 1;

FIG. 5 is a longitudinal, cross-sectional view of another end effector assembly configured for use with the forceps of FIG. 1 or the system of FIG. 3;

FIG. 6 is a transverse, cross-sectional view of the end effector assembly of FIG. 5 with a sensor(s) separate from the end effector assembly;

FIG. 7 is a block diagram of a generator configured for use with the end effector assemblies of FIGS. 5 and 6;

FIG. 8 is a flow diagram illustrating a method of sealing tissue in accordance with the present disclosure;

FIG. 9A is a flow diagram illustrating a method of sealing tissue in accordance with the present disclosure wherein pre-treatment is always implemented;

FIG. 9B is a flow diagram illustrating a method of sealing tissue in accordance with the present disclosure wherein sensor-based feedback is utilized to determine if pre-treatment is to be performed;

FIG. 9C is a flow diagram illustrating a method of sealing tissue in accordance with the present disclosure wherein a user-selection dictates whether pre-treatment is performed; and

FIG. 10 is a plot of experimental results of burst pressure for various vessel sizes sealed using a control algorithm compared with an algorithm of the present disclosure.

### DETAILED DESCRIPTION

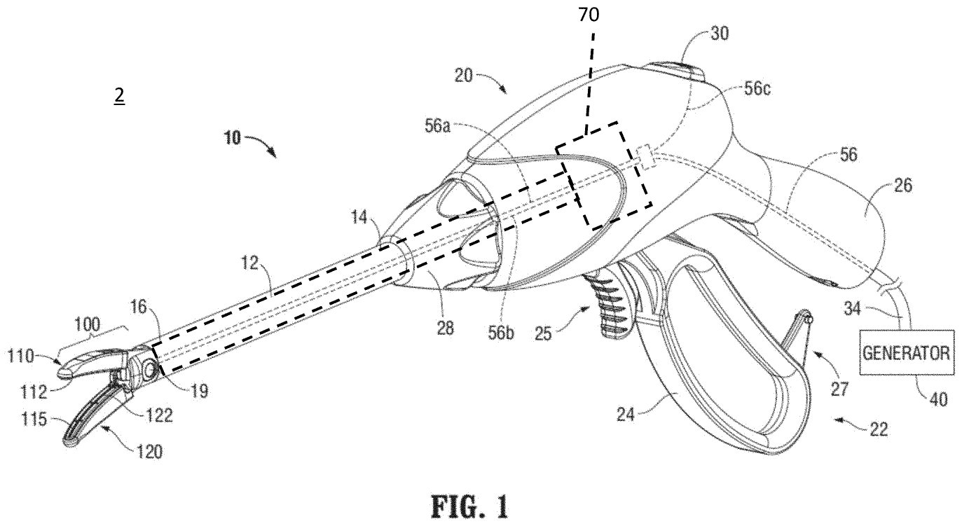

Referring to FIG. 1, an electrosurgical system in accordance with the present disclosure is shown generally identified by reference numeral 2. Electrosurgical system 2 includes an electrosurgical forceps 10 and an electrosurgical generator 40. Electrosurgical forceps 10 is shown and described herein as a shaft-based, manual device. However, any other suitable electrosurgical forceps, whether shaft-based, hemostat-style, manual, partly powered, fully powered, robotic, etc. may be utilized in accordance with the present disclosure. Obviously, different connections and considerations apply to each particular type of instrument; however, the aspects and features of the present disclosure with respect to sealing tissue remain generally consistent with respect to any suitable instrument.

Continuing with reference to FIG. 1, forceps 10 includes a shaft 12, a housing 20, a handle assembly 22, a trigger assembly 25, a rotating assembly 28, and an end effector assembly 100. Shaft 12 has a distal end portion 16 configured to mechanically engage end effector assembly 100 and a proximal end portion 14 that mechanically engages housing 20. A cable 34 couples forceps 10 to electrosurgical generator 40 for transmitting energy and control signals between generator 40 and forceps 10. Cable 34 houses a plurality of wires 56 that are internally divided within handle assembly 22 and/or in shaft 12 into wires 56a-56c, which electrically interconnect end effector assembly 100, activation switch 30, and/or generator 40 with one another.

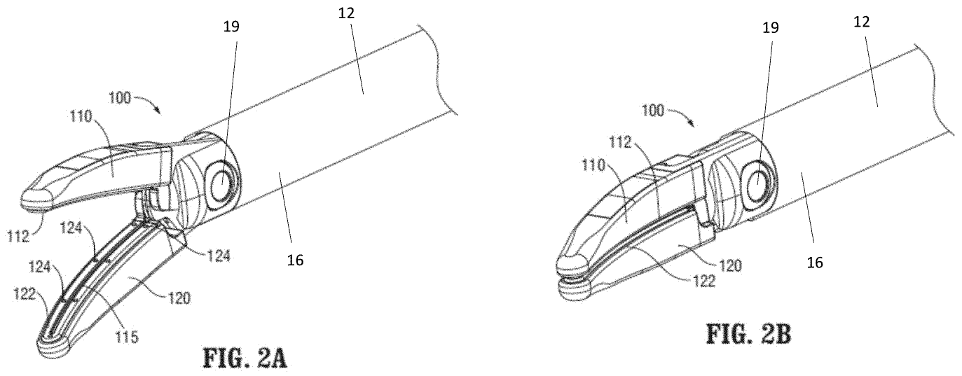

Handle assembly 22 includes a movable handle 24 and a fixed handle 26. Fixed handle 26 is integrally associated with housing 20 and movable handle 24 is movable relative to fixed handle 26. Movable handle 24 is ultimately connected to a drive assembly 70 that, together, mechanically cooperate to impart movement of one or both of jaw members 110, 120 of end effector assembly 100 relative to the other between a spaced-apart position and an approximated position to grasp tissue therebetween. As shown in FIG. 1, movable handle 24 is initially spaced-apart from fixed handle 26 and, correspondingly, jaw members 110, 120 are disposed in the spaced-apart position (see also FIG. 2A). Movable handle 24 is movable from this initial position to one or more compressed positions corresponding to one or more approximated positions of jaw members 110, 120 (see FIG. 2B).

Drive assembly 70 may be configured to regulate the clamping force applied to tissue grasped between surfaces 112, 122 of jaw members 110, 120, respectively. More specifically, handle assembly 22 and/or latching assembly 27, in conjunction with drive assembly 70, may be configured such that jaw members 110, 120 impart a specific clamping force or clamping force within a specific clamping force range to tissue grasped between surfaces 112, 122 of jaw members 110, 120, respectively. This may be achieved manually, e.g., via moving movable handle 24 from the initial position to a specific compressed position (or positions), e.g., a fully compressed position; via mechanical latching, e.g., wherein a latch assembly 27 is configured to latch movable handle 24 in a specific position (or positions); via a powered actuator with feedback-based control, e.g., via driving or reversing a motor-controlled actuator to a specific position (or positions); and/or via any other suitable mechanism. Drive assembly 70, in any of the configurations detailed above or any other suitable configuration, may include one or more passive regulating components, e.g., springs, resilient features, etc., and/or active regulating components, e.g., motor(s), manual drives, etc.

Suitable mechanisms for use as or in conjunction with drive assembly 70 for clamping force control include those described in U.S. Pat. Nos. 5,776,130; 7,766,910; 7,771,426; and 8,226,650; and/or U.S. Patent Application Pub. Nos. 2009/0292283; 2012/0172873; and 2012/0184988, the entire contents of all of which are hereby incorporated by reference herein. Other suitable mechanisms for applying a specific clamping force or clamping force within a specific clamping force range to tissue grasped between jaw members 110, 120 may also be provided. With tissue grasped between jaw members 110, 120 under the specific clamping force or clamping force within a specific clamping force range, energy may be supplied to either or both tissue contacting surfaces 112, 122 of jaw members 110, 120, respectively, to seal tissue, e.g., via activation of activation switch 30.

The jaw clamping force, measured at a midpoint along the lengths of jaw members 110, 120, may be in a range of (or the jaw force range may be) from about 7.0 lbf to about 11.0 lbf; in other aspects from about 8.0 lbf to about 10.0 lbf; and, in still other aspects, from about 8.5 lbf to about 9.5 lbf.

Latching assembly 27 may be provided for selectively locking movable handle 24 relative to fixed handle 26 at various positions between the initial position and the compressed position(s) to correspondingly lock jaw members 110, 120 at various different positions during pivoting, e.g., the one or more approximated positions. Rotating assembly 28 is rotatable in either direction to similarly rotate shaft 12 and end effector assembly 100 relative to housing 20.

Referring also to FIGS. 2A and 2B, end effector assembly 100 is shown attached at the distal end portion 16 of shaft 12 and includes opposing jaw members 110 and 120. Each jaw member 110, 120 includes an electrically conductive tissue contacting surface 112, 122, respectively, that cooperate to grasp tissue therebetween, e.g., in the one or more approximated positions of jaw members 110, 120, and to facilitate sealing the grasped tissue via conducting the energy from generator 40 therebetween. More specifically, tissue contacting surfaces 112, 122 are electrically coupled to generator 40, e.g., via wires 56a, 56b, and are configured to be energized to different potentials to enable the conduction of Radio Frequency (RF) electrosurgical energy provided by generator 40 between tissue contacting surfaces 112, 122 and through tissue grasped therebetween to seal tissue. Tissue contacting surfaces 112, 122 may be defined by electrically conductive plates secured to jaw members 110, 120, may be defined by surfaces of jaw members 110, 120 themselves, may be formed via the deposition of material onto jaw members 110, 120, or may be defined and/or formed in any other suitable manner.

Either or both jaw members 110, 120 may further include one or more stop members 124 (FIG. 2A) disposed on or otherwise associated with either or both tissue-contacting surface 112, 122 to maintain a minimum gap distance between tissue contacting surfaces 112, 122 when jaw members 110, 120 are disposed in a fully approximated position, thus inhibiting electrical shorting. Stop members 124 may be insulative, partly insulative, and/or electrically isolated from either or both tissue contacting surfaces 112, 122. In aspects, in the approximated position of jaw members 110, 120, it is desirable to maintain a gap distance within a suitable gap distance range to ensure consistent and effective tissue sealing. The gap distance may be controlled by stop members 124, movable handle 24, latching assembly 27, and/or drive assembly 70, and, in aspects, may be from about 0.001 inches to about 0.010 inches; in other aspects from about 0.001 inches to about 0.008 inches; and, in still other aspects form about 0.001 inches to about 0.006 inches. Other suitable gap distance ranges are also contemplated. The gap distance may be determined as the maximum gap distance between the tissue contacting surfaces 112, 122 at any point therealong.

An activation switch 30 is disposed on housing 20 and is coupled between or otherwise to generator 40 and/or tissue-contacting surfaces 112, 122 via wire 56c. Activation switch 30 is selectively activatable to initiate the supply of energy from generator 40 to tissue contacting surfaces 112, 122 of jaw members 110, 120, respectively, of end effector assembly 100. More specifically, depression of activation switch 30 is recognized, e.g., as a resistance drop, by generator 40 to signal to generator 40 to initiate tissue sealing, e.g., to supply energy to jaw members 110, 120.

End effector assembly 100 is designed as a bilateral assembly, e.g., wherein both jaw member 110 and jaw member 120 are movable about a pivot 19 relative to one another and to shaft 12. However, end effector assembly 100 may alternatively be configured as a unilateral assembly, e.g., wherein one of the jaw members 110, 120 is fixed relative to shaft 12 and the other jaw member 110, 120 is movable about pivot 19 relative to shaft 12 and the fixed jaw member.

In some configurations, a knife assembly (not shown) is disposed within shaft 12 and a knife channel 115 is defined within one or both jaw members 110, 120 to permit reciprocation of a knife blade (not shown) therethrough, e.g., via actuation of trigger assembly 25, to mechanically cut tissue grasped between jaw members 110, 120. In aspects, the knife blade is energizable to enable dynamic energy-based tissue cutting. Alternatively, end effector assembly 100 may include a static energy-based tissue cutter (not shown), e.g., disposed one or within one of the jaw members 110, 120. The energy-based cutter, whether static or dynamic, may be configured to supply any suitable energy, e.g., RF, microwave, infrared, light, ultrasonic, thermal, etc., to tissue for energy-based tissue cutting. Energy activation for tissue cutting may be initiated via trigger assembly 25, automatically after tissue sealing, via a different (or further) activation of switch 30, via a separate actuation button, via a foot switch (not shown), or in any other suitable manner.

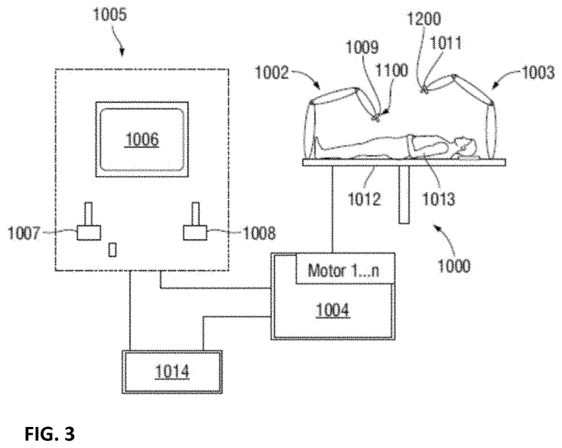

Turning to FIG. 3, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in an operating mode. Robotic surgical instrument 1000 may be configured for use on a patient 1013 lying on a patient table 1012 to be treated in a minimally invasive manner. Robotic surgical instrument 1000 may further include or be capable of accessing a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100, for example, may be similar to and include any of the features of end effector assembly 100 (FIGS. 1-2B) and, together with robot arm 1002, functions similarly as detailed above with respect to forceps 10 except in a robotically-actuated and controlled manner. Other suitable end effector assemblies for coupling to attaching device 1009 are also contemplated. End effector assembly 1200 may be any end effector assembly, e.g., a surgical camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

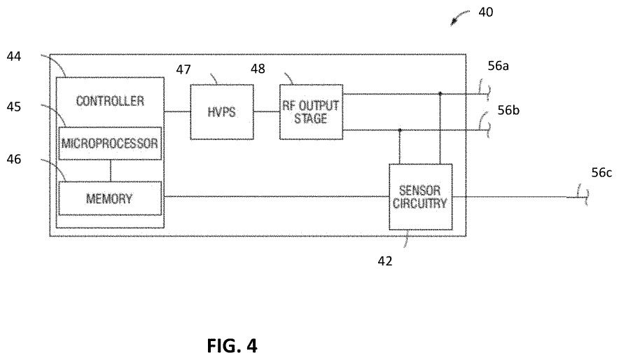

With reference to FIG. 4, generator 40 may be configured for use with forceps 10 (FIG. 1), robotic surgical system 1000 (FIG. 3), and/or any other suitable surgical instrument or system. Generator 40 includes sensor circuitry 42, a controller 44, a high voltage DC power supply (“HVPS”) 47 and an RF output stage 48. HVPS 47 provides high voltage DC power to RF output stage 48 which converts the high voltage DC power into RF energy for delivery to the end effector assembly, e.g., tissue-contacting surfaces 112, 122 of jaw members 110, 120, respectively, of end effector assembly 100 (FIGS. 1-2B). In particular, RF output stage 48 generates sinusoidal waveforms of high frequency RF energy. RF output stage 48 is configured to generate a plurality of waveforms having various duty cycles, peak voltages, crest factors, and other parameters, depending on a particular mode of operation.

Controller 44 includes a microprocessor 45 operably connected to a memory 46 which may be volatile type memory (e.g., RAM) and/or non-volatile type memory (e.g., flash media, disk media, etc.). Microprocessor 45 is operably connected to HVPS 47 and/or RF output stage 48 allowing microprocessor 45 to control the output of generator 40, e.g., in accordance with feedback received from sensor circuitry 42. Sensor circuitry 42 is operably coupled to wires 56a, 56b, which supply energy to/from tissue-contacting surfaces 112, 122 (FIGS. 1-2B). From wires 56a, 56b and, more specifically, the signals transmitted therealong, sensor circuitry 42 may determine one or more parameters, e.g., tissue impedance, output current and/or voltage, etc. Sensor circuitry 42 provides feedback, e.g., based on the sensed parameter(s), to controller 44 which, in turn, selects an energy-delivery algorithm, modifies an energy-delivery algorithm, and/or adjusts energy-delivery parameters based thereon. Sensor circuitry 42 or controller 44 may also monitor wire 56c to determine activation (and/or deactivation) of switch 30 (FIG. 1) to, in response thereto, initiate (or terminate) the supply of energy based thereon.

FIG. 5 illustrates another end effector assembly provided in accordance with the present disclosure, shown generally identified by reference numeral 300. End effector assembly 300 is similar to end effector assembly 100 (FIGS. 1-2B) and may include any of the features thereof. End effector assembly 300 may be utilized with forceps 10 (FIG. 1), robotic surgical system 1000 (FIG. 3), or any other suitable surgical instrument or system. End effector assembly 300 includes first and second jaw members 310, 320 each including a respective electrically conductive tissue contacting surface 312, 322. Either or both of jaw members 310, 320 is movable relative to the other between a spaced-apart position and one or more approximated positions to grasp tissue between tissue contacting surfaces 312, 322. Wires 314, 324 electrically couple tissue contacting surfaces 312, 322 to a source of energy, e.g., generator 400 (FIG. 7), for conducting RF energy between tissue contacting surfaces 312, 322 and through tissue grasped therebetween to seal tissue.

Either or both jaw members 310, 320 of end effector assembly 300 further includes a sensor 316, 326 positioned thereon or therein. Sensors 316, 326 may be any suitable sensors configured to sense one or more parameters, may sense similar or different parameters, and/or may operate independently or collectively. Sensors 316, 326 may be configured as, for example: electrical sensors, e.g., configured to sense impedance, current, power, voltage, etc.; optical sensors, e.g., configured to sense one or more optical properties of end effector assembly 300, tissue, and/or the surrounding environment such as, for example, color, clarity, transparency, etc.; distance (linear or angular) sensors configured to determine a linear distance or angle between jaw members 310, 320 and/or other components; proximity sensors, e.g., hall effect sensors, configured to determine a distance between jaw members 310, 320 and/or other components to, for example, enable determination of physical tissue properties (diameter, mass, density, compressibility, etc.); particle sensors, e.g., an ionization detector or a photoelectric detector, configured to detect smoke and/or other particles; electronic nose sensors configured to electronically sense one or more smell-based properties of tissue and/or the surrounding environment; chemical sensors, e.g., a molecular sensor, gas chromatograph, etc., configured to sense one or more chemical properties of tissue and/or the surrounding environment; moisture sensors; pressure/force sensors; density sensors; temperature and/or thermal sensors; ultrasonic sensors; audio sensors; etc. Further, combinations of sensors, e.g., two or more of the above-noted or other suitable sensors, may be utilized.

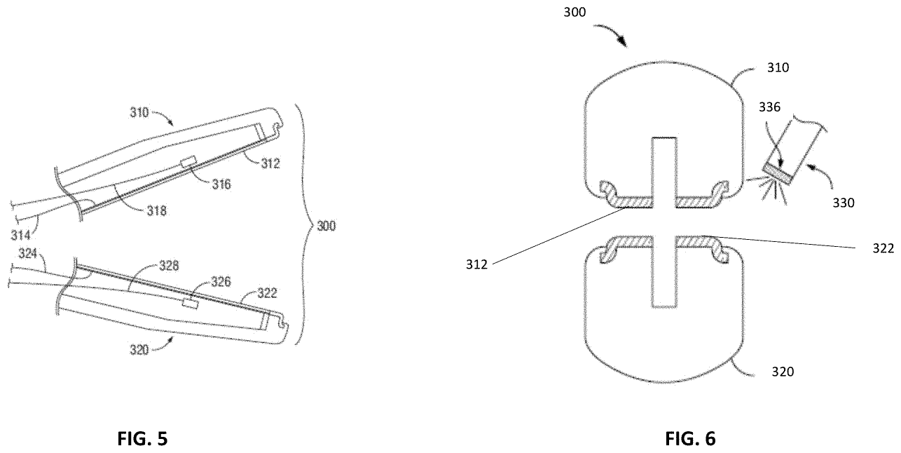

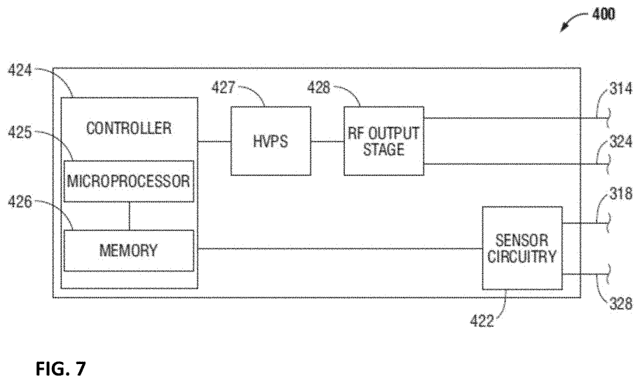

Alternatively, as shown in FIG. 6, one or more sensors 336, configured similar to any of the sensors noted above or any other suitable sensor, may be disposed on a device 330 separate from end effector assembly 300 and configured to sense one or more properties of end effector assembly 300, of tissue in contact therewith, e.g., grasped thereby, and/or of the surrounding environment. In aspects, sensor 336 is a visible image surgical camera configured to sense the position and/or angle between jaw members 310, 320. The sensor 336 may alternatively be configured as an infrared camera, thermal camera, or other suitable camera or other sensor.

Referring to FIGS. 5-7, another generator 400 provided in accordance with the present disclosure is similar to generator 40 (FIG. 4) and may include any of the features thereof. Generator 400 is configured for use with end effector assembly 300 and, similarly as with generator 40 (FIG. 4), includes sensor circuitry 422, a controller 424 (including a microprocessor 425 and memory 426), an HVPS 427 and an RF output stage 428. Generator 400 is configured to supply energy to end effector assembly 300 via wires 314, 324. Sensor circuitry 422 is operably coupled to wires 318, 328 (and/or wire(s) or other wired or wireless connections to sensor 336) and is thereby configured to receive the sensed parameters from sensors 316, 326 (and sensor 336). Sensor circuitry 422 provides feedback, e.g., based on the sensed parameter(s), to controller 424 which, in turn, selects an energy-delivery algorithm, modifies an energy-delivery algorithm, and/or adjusts energy-delivery parameters based thereon.

With general reference to FIGS. 1-7, sealing tissue, e.g., blood vessels, tissue including blood vessels, other tissue structures, etc., is accomplished by controlling both mechanical and electrical factors. More specifically, with respect to the mechanical factors, achieving effective and consistent tissue seals is facilitated by controlling the gap distance between the opposing electrically-conductive surfaces grasping tissue therebetween and by controlling the clamping force applied to the grasped tissue. With respect to the electrical factors, controlling the intensity, frequency, and duration of the electrosurgical energy applied to the clamped tissue are important electrical considerations for sealing tissue.

Controlling the mechanical factors associated with tissue sealing for some tissue, e.g., some tissue types, conditions, and/or tissue sizes (e.g., blood vessel diameters or tissue structure diameters less than or equal to about 7.0 mm), the electrosurgical instrument (such as any of the instruments, end effector assemblies, or systems detailed hereinabove) itself may enable control of the mechanical factors within suitable ranges. More specifically, the instrument may be configured, in an approximated position of the jaw members, to control the clamping force to within an appropriate clamping force range (such as those detailed above) and to control the gap distance to within an appropriate gap distance range (such as those detailed above). In such instances, a tissue sealing algorithm may be implemented for controlled delivery of energy to achieve effective and consistent tissue seals.

Controlling the mechanical factors for other tissue, e.g., some tissue types, conditions, and/or tissue sizes (e.g., blood vessel diameters or tissue structure diameters equal to or greater than about 7.1 mm), may not be capable of being consistently and effectively accomplished solely by the mechanical configuration of the electrosurgical instrument. Rather, in such instances, it may be necessary to pre-treat tissue, e.g., by implementing a pre-treat algorithm, in order to shrink the tissue, make the tissue more compressible, or otherwise modify the tissue such that the electrosurgical instrument is capable of achieving a clamping force and/or gap distance within the appropriate ranges thereof. Alternatively or additionally, the pre-treatment may modify the tissue in such a manner that the appropriate clamping force and/or gap distance ranges suitable for sealing the tissue are altered, e.g., enlarged, to ranges capable of being achieved by the electrosurgical instrument acting on the pre-treated tissue. Pre-treatment may also be desired in other situations (whether or not the mechanical factors may be adequately controlled), depending upon the conditions of the procedure, the tissue type, tissue condition, tissue size, etc. Once the pre-treat algorithm is completed and/or pre-treatment is determined to be completed, a tissue sealing algorithm may be implemented for controlled delivery of energy to achieve effective and consistent tissue seals.

Determining whether tissue may require pre-treatment to facilitate tissue sealing may be based on a manual input by a user during the procedure, e.g., activation of a switch on the instrument, button on the generator, etc., or prior thereto based on, for example, obtained pre-operative information such as patient data, the procedure to be performed, etc. Alternatively or additionally, determining whether tissue may require pre-treatment to facilitate tissue sealing may be based on sensed parameters, e.g., from the sensors and/or sensor circuitry according to any of the aspects detailed above or in any other suitable manner.

For example, distance sensors, proximity sensors, and/or surgical cameras may be utilized to enable determination of a size of tissue based upon the distance(s) and/or angle between the jaw members, based on the clamping force applied, and/or based on the movement, position, and/or force associated with other mechanical components coupled to the jaw members. Jaw angle, more specifically, may be determined as detailed in U.S. Pat. No. 8,357,158, titled “JAW CLOSURE DETECTION SYSTEM” and filed as U.S. patent application Ser. No. 12/419,735 on Apr. 7, 2009, the entire contents of which are hereby incorporated herein by reference; jaw pressure/force, more specifically, may be determined as detailed in U.S. Pat. No. 10,695,123, titled “SURGICAL INSTRUMENT WITH SENSOR” and filed as U.S. patent application Ser. No. 15/401,227 on Jan. 9, 2017, the entire contents of which are hereby incorporated herein by reference; and jaw aperture/angle, more specifically, may be determined as detailed in U.S. Patent Application Publication No. 2017/0215944, titled “JAW APERTURE POSITION SENSOR FOR ELECTROSURGICAL FORCEPS” and filed as U.S. patent application Ser. No. 15/418,809 on Jan. 30, 2017, the entire contents of which are hereby incorporated herein by reference. Where the determined tissue diameter (based on, e.g., jaw angle, jaw pressure/force, etc.) or other determined tissue property exceeds an upper or lower threshold limit (depending upon the particular property in question), it may be determined that pre-treatment is needed; on the other hand, when the determined property does not exceed the threshold limit, it may be determined that pre-treatment is not needed.

As another example, determination of tissue type or condition, for example, may be made via sensing electrical properties of tissue, physical properties of tissue, visual identification, chemical analysis, optical analysis, combinations thereof, and/or in in any other suitable manner. When certain types of tissue or conditions of tissue are identified, it may be determined that pre-treatment is needed; on the other hand, when other types of tissue or conditions are identified, it may be determined that pre-treatment is not needed.

Any or all of the above tissue type, condition, and/or tissue size determinations may be facilitated, together with sensor data or without such sensor data, by the use of one or more algorithms, set points, look-up tables, machine learning programs, etc. of which the stored or training data is obtained experimentally, via mathematical simulation, utilizing machine learning, using theoretical formulae, combinations thereof, etc. Likewise, determining whether the identified tissue properties, type, condition, and/or tissue size requires pre-treatment may be facilitated by the use of one or more algorithms, set points, look-up tables, machine learning programs, etc. of which the stored or training data is obtained experimentally, via mathematical simulation, utilizing machine learning, using theoretical formulae, combinations thereof, etc.

Determining the need for a pre-treatment may also be initiated in response to a generator error during an attempt to seal tissue without the pre-treatment such as, for example, an alarm indicating that the grasped tissue cannot be sealed and that the user should re-grasp tissue. In such instances, a subsequent activation of the activation switch (with or without re-grasp) may initiate the pre-treat algorithm prior to implementing the sealing algorithm.

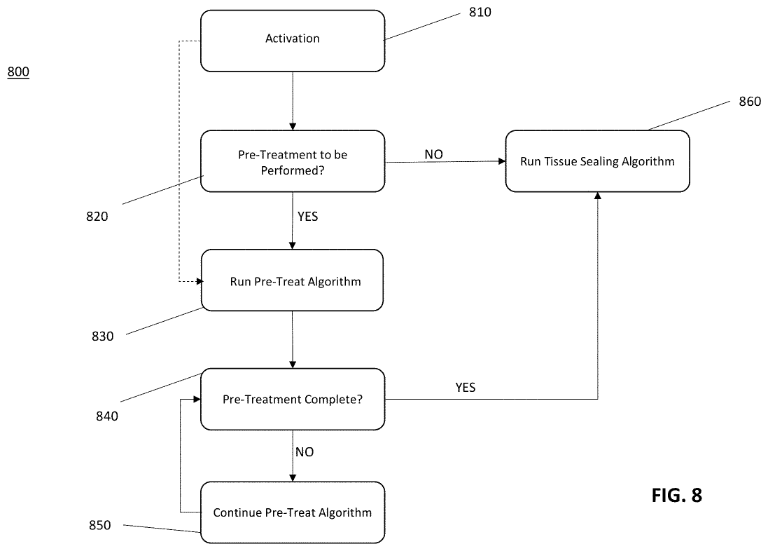

Turning to FIG. 8, a method of sealing tissue using an electrosurgical instrument is provided in accordance with the present disclosure and shown generally identified by reference numeral 800. Method 800 begins at 810 when the electrosurgical instrument is activated, e.g., when activation switch 30 is activated with tissue grasped between jaw members 110, 120 (FIG. 1).

When the electrosurgical instrument is activated, it is determined at 820 whether pre-treatment is to be performed. Whether or not pre-treatment is determined to be performed may be based on, as detailed above, a manual input; based on tissue type, condition, and/or tissue size, e.g., using sensor feedback; or in any other suitable manner. Where it is determined at 820 that pre-treatment is to be performed (“YES” at 820), a pre-treat algorithm is initiated at 830. As an alternative to determining whether pre-treatment is to be performed at 820, the method may skip from activation at 810 to initiating the pre-treat algorithm at 830. Pre-treatment may automatically be performed, for example, in all cases or based on activation of a pre-treat setting associated with the instrument or generator.

The pre-treat algorithm may be a voltage-controlled algorithm or any other suitable algorithm, e.g., power-controlled, impedance-controlled, current-controlled, time-controlled, combinations thereof, etc. The pre-treat algorithm may continue until an end of the pre-treat algorithm is determined, which may be impedance-based, time-based, voltage-based, power-based, current-based, and/or sensor feedback-based (e.g., wherein a threshold size is reached, a threshold temperature is reached, a threshold color is achieved, etc.) Where multiple end indicators are utilized, the pre-treat algorithm may end when the first in time indicator is reached, only after all indicators are reached, or in any other suitable manner.

The pre-treat algorithm, more specifically, may be voltage-controlled that is, supplying energy at an initial voltage and modifying the voltage in accordance with a voltage versus time graph, e.g., according to a linear voltage ramp, a polynomial function, or in any other suitable manner. For example, the initial voltage may start at about 50 V and ramp linearly at a rate of about 5 V per second. In other aspects, the initial voltage may start at about 25V and ramp linearly at a rate of about 20V per second. The initial voltage, in aspects, may be from about 20V to about 80V and/or the ramp may be from about 3 V per second to about 25 V per second. Other initial voltages and/or voltage ramps are also contemplated. For example, depending upon the size and/or configuration of the jaw members, the appropriate voltage(s) may need to be modified.

The end of the pre-treat algorithm may be determined, for example, after a pre-determined change in impedance of tissue between an initial impedance and an end impedance. The pre-determined impedance change may be, in aspects, up to about 100 ohms; in other aspects up to about 150 ohms, and in still other aspects, up to about 200 ohms. As noted above, the end of the pre-treat algorithm may additionally or alternatively be based on sensor feedback such as, for example, when a gap distance within the appropriate gap distance range is achieved, when a clamping force within the appropriate clamping force range is achieved, at the end of a pre-determined time period, etc. It is determined, at 840, whether the pre-treat algorithm has been completed, e.g., according to any one or more of the end determinations above. If the pre-treat algorithm is determined to have been completed (“YES” at 840), the method proceeds to 850, where the tissue sealing algorithm is initiated. If the pre-treat algorithm is determined to not have been completed (“NO” at 840), running the pre-treat algorithm continues at 850 until the end is determined.

Where the end of the pre-treat algorithm is reached (“YES” at 840), or where it is determined that pre-treatment is not to be performed (“NO” at 820), the method proceeds to 860, where the tissue sealing algorithm is initiated. In aspects, a delay period may occur between the pre-treatment algorithm and tissue sealing algorithm, e.g., of from about 10 milliseconds to about 2 seconds, although other delay periods are also contemplated. The tissue sealing algorithm may be independent from the pre-treat algorithm and based on a different or the same control scheme. For example, tissue sealing algorithm may be impedance-controlled or may utilize any other suitable control, e.g., voltage control, power control, current control, time control, combinations thereof, etc. The tissue sealing algorithm may include multiple stages utilizing different control and/or different parameters. An exemplary tissue sealing algorithm is detailed below, although any other suitable tissue sealing algorithm may be utilized.

The tissue sealing algorithm may begin with an impedance sense phase during which the algorithm senses the tissue impedance with an interrogatory impedance sensing pulse. Tissue impedance is determined without appreciably changing the tissue during this phase. During this interrogation or error-checking phase, constant power is provided to check for a short or an open circuit, in order to determine if tissue is grasped. If no short or open circuit is detected, the algorithm proceeds. If a short or open circuit is detected, an error is returned. In aspects, this interrogatory phase may be skipped in instances where pre-treatment has been performed; in aspects, this interrogatory phase may be performed at the onset of pre-treatment as an alternative or in addition to being performed at the onset of the tissue sealing algorithm.

If no short or open circuit is detected, the algorithm initiates the supply of electrosurgical energy to tissue at an initial output level or in accordance with and initial control, e.g., delivering current linearly over time to heat the tissue. During this energy output, it is determined whether a tissue reaction, e.g., reaching a boiling point of tissue fluid, has occurred as a function of a minimum impedance value and a predetermined rise in impedance. A target impedance trajectory, e.g., including a plurality of target impedance values, as a function of measured impedance and a desired rate of change based on the tissue reaction determination is then generated and the output energy is adjusted to substantially match tissue impedance to the corresponding target impedance values, thereby following the target impedance trajectory. The output energy may be controlled until an ending impedance value is reached, at which point energy is terminated. A cooling period may ensue prior to indicating that tissue sealing is complete, e.g., with an audible and/or visual tone.

In aspects, rather than or in addition to implementing the pre-treatment algorithm prior to the tissue sealing algorithm, the tissue sealing algorithm may be interrupted at a pre-determined, sensed feedback-based, or user-input point and the pre-treatment algorithm may then be executed prior to resuming the tissue sealing algorithm to completion. In aspects where the tissue sealing algorithm includes multiple phases, the pre-treatment algorithm may be performed in between any suitable phases thereof. In still other aspects, the pre-treatment algorithm may additionally or alternatively be performed after the tissue sealing algorithm.

More detailed tissue sealing algorithms suitable for use in accordance with the present disclosure can be found in, for example, U.S. Pat. No. 8,920,421, titled “SYSTEM AND METHOD FOR TISSUE SEALING” and filed as U.S. patent application Ser. No. 12/995,042 on Nov. 29, 2010, the entire contents of which are hereby incorporated herein by reference; and U.S. Pat. No. 8,147,485, titled “SYSTEM AND METHOD FOR TISSUE SEALING” and filed as U.S. patent application Ser. No. 12/391,036 on Feb. 23, 2009, the entire contents of which are hereby incorporated herein by reference.

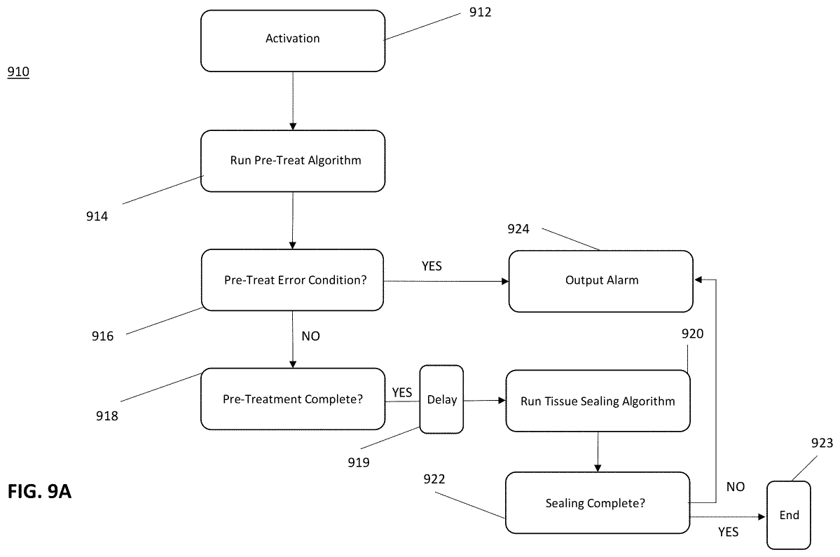

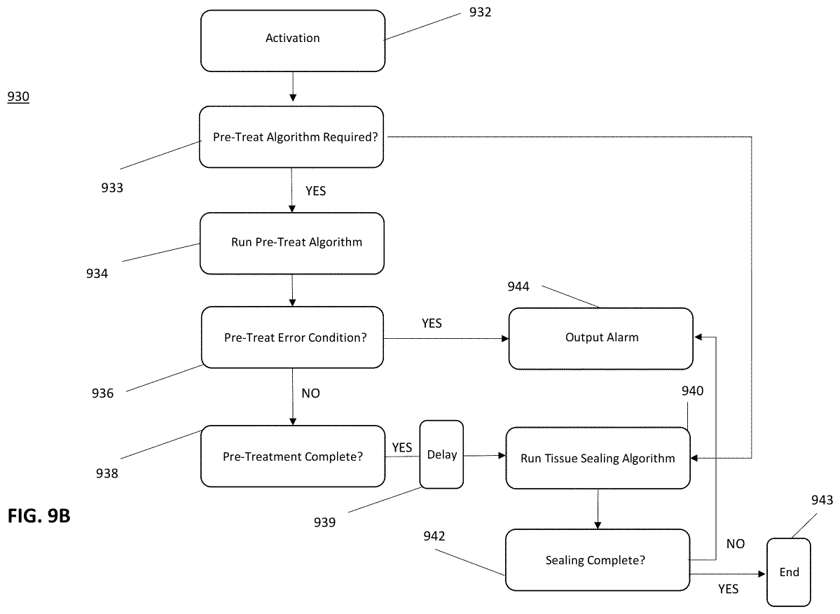

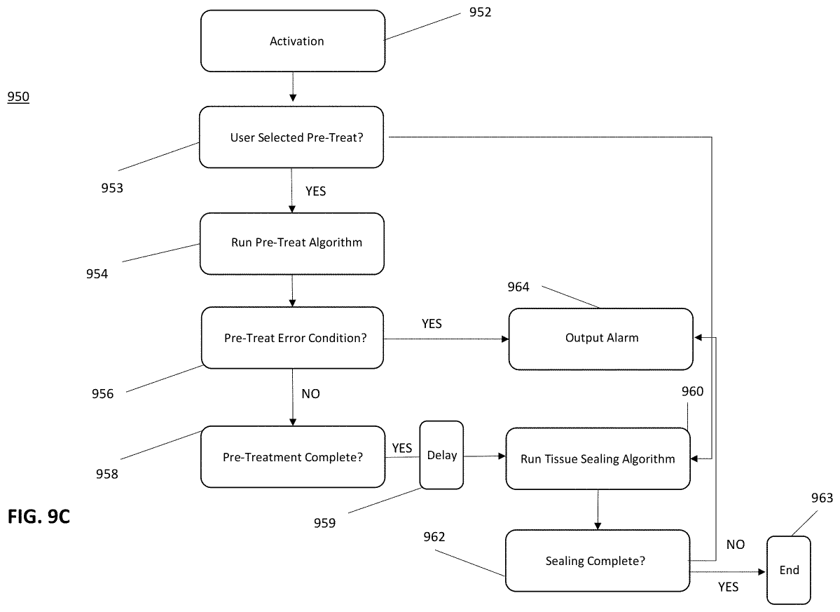

With reference to FIGS. 9A-9C, methods of sealing tissue specific to an always pre-treat configuration, a sensor-based pre-treat configuration, and a user-input-based pre-treat configuration, respectively, are detailed. However, other suitable configurations are also contemplated. Aspects and features detailed above or otherwise herein are not repeated in detail to avoid unnecessary repetition.

Turning to FIG. 9A, with respect to an always pre-treat configuration, method 910 starts at 912 upon activation, e.g., when activation switch 30 is activated with tissue grasped between jaw members 110, 120 (FIG. 1). The method continues to 914 wherein the pre-treat algorithm is run and, during the pre-treat algorithm, it is determined whether there are any error conditions at 916, e.g., whether a short circuit is detected, where there is no impedance change or an insufficient impedance change, etc. If an error condition is detected, “YES” at 916, the pre-treat algorithm is terminated and an alarm is output at 924. If no error condition is detected, “NO” at 916, the method continues. Also during the running of the pre-treat algorithm, it is determined, at 918, if pre-treatment is complete, e.g., in any of the manners detailed above or in any other suitable manner. If pre-treatment is not complete, pre-treatment continues and it is continually or periodically determined whether pre-treatment is compete (at 918) and/or whether an error condition is detected (at 916). In other words, 916 and 918 are repeated simultaneously, consecutively, or in any other suitable manner during running of the pre-treat algorithm.

When pre-treatment is complete, “YES” at 918, a delay is instituted at 919, before initiating the tissue sealing algorithm at 920. The tissue sealing algorithm continues until sealing is determined to be complete. If complete, “YES” at 922, the method ends at 923. If not complete, “NO” at 922, or when an error condition is detected, the tissue sealing algorithm is terminated and an alarm is output at 924.

Referring to FIG. 9B, with respect to a sensor-based pre-treat configuration, method 930 starts at 932 upon activation, e.g., when activation switch 30 is activated with tissue grasped between jaw members 110, 120 (FIG. 1). The method continues to 933 where it is determined, e.g., based on the sensor feedback (such as, for example, detailed above), if pre-treatment is to be performed. If pre-treatment is determined to be required, “YES” at 933, the method proceeds to 934 wherein the pre-treat algorithm is run and, during the pre-treat algorithm, it is determined whether there are any error conditions at 936. If an error condition is detected, “YES” at 936, the pre-treat algorithm is terminated and an alarm is output at 944. If no error condition is detected, “NO” at 936, the method continues. Also during the running of the pre-treat algorithm, it is determined, at 938, if pre-treatment is complete. If pre-treatment is not complete, pre-treatment continues and it is continually or periodically determined whether pre-treatment is compete (at 938) and/or whether an error condition is detected (at 936).

When pre-treatment is complete, “YES” at 938, a delay is instituted at 939, before initiating the tissue sealing algorithm at 940. The tissue sealing algorithm continues until sealing is determined to be complete. If complete, “YES” at 942, the method ends at 943. If not complete, “NO” at 942, or when an error condition is detected, the tissue sealing algorithm is terminated and an alarm is output at 944.

With reference to FIG. 9C, with respect to a user-input-based pre-treat configuration, method 950 starts at 952 upon activation, e.g., when activation switch 30 is activated with tissue grasped between jaw members 110, 120 (FIG. 1). The method continues to 953 where it is determined if the user has or had selected pre-treatment. If pre-treatment is/was selected, “YES” at 953, the method proceeds to 954 wherein the pre-treat algorithm is run and, during the pre-treat algorithm, it is determined whether there are any error conditions at 956. If an error condition is detected, “YES” at 956, the pre-treat algorithm is terminated and an alarm is output at 964. If no error condition is detected, “NO” at 956, the method continues. Also during the running of the pre-treat algorithm, it is determined, at 958, if pre-treatment is complete. If pre-treatment is not complete, pre-treatment continues and it is continually or periodically determined whether pre-treatment is compete (at 958) and/or whether an error condition is detected (at 956).

When pre-treatment is complete, “YES” at 958, a delay is instituted at 959, before initiating the tissue sealing algorithm at 960. The tissue sealing algorithm continues until sealing is determined to be complete. If complete, “YES” at 962, the method ends at 963. If not complete, “NO” at 962, or when an error condition is detected, the tissue sealing algorithm is terminated and an alarm is output at 964.

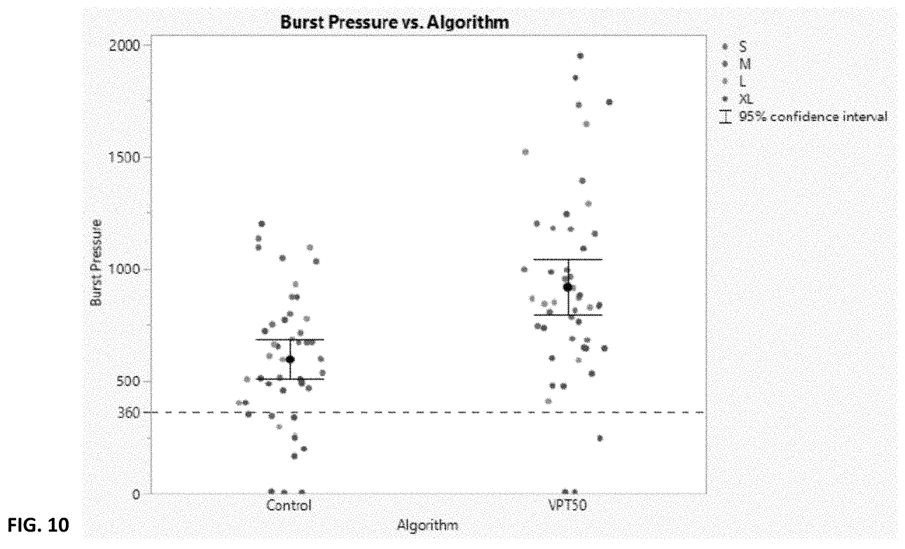

Referring to FIG. 10, a plot of experimental results of burst pressure for various vessel sizes sealed using a control algorithm compared with an algorithm of the present disclosure is shown. The indicted vessel sizes are small, “S,” up to about 3.0 mm in diameter; medium, “M,” from about 3.1 mm to about 5.0 mm in diameter; large, “L,” from about 5.1 mm to about 7.0 mm; and extra large, “XL,” from about 7.1 mm to about 10.0 mm. Burst pressure is shown in units of mmHg. The control algorithm is provided as a tissue sealing algorithm, e.g., in accordance with the present disclosure, without the use of a pre-treatment algorithm. The VPT50 algorithm includes the same tissue sealing algorithm as the control algorithm but implements the pre-treatment algorithm prior thereto. As can be appreciated by viewing these experimental results, the burst pressure levels (particularly for large and extra large vessels) as well as the 95% confidence interval level is increased using the VPT50 algorithm compared to the control algorithm.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular configurations. Those skilled in the art will envision other modifications within the scope and spirit of the claims appended hereto.

What is claimed is:

1. A method of sealing tissue, comprising:
grasping tissue between first and second jaw members;
applying energy to the grasped tissue in accordance with a pre-treatment algorithm to pre-treat the grasped tissue in anticipation of tissue sealing; and
applying energy to the pre-treated, grasped tissue in accordance with a tissue sealing algorithm to seal the pre-treated grasped tissue.

2. The method according to claim 1, wherein the tissue sealing algorithm is independent of the pre-treatment algorithm.

3. The method according to claim 1, wherein the pre-treatment algorithm is controlled in a first manner and the tissue sealing algorithm is controlled in a second, different manner.

4. The method according to claim 1, wherein at least a portion of the pre-treatment algorithm is voltage-controlled.

5. The method according to claim 4, wherein at least a portion of the pre-treatment algorithm follows a voltage versus time graph.

6. The method according to claim 5, wherein the voltage versus time graph includes a fixed positive voltage ramp.

7. The method according to claim 1, wherein at least a portion of the tissue sealing algorithm adjusts energy output to track an impedance versus time trajectory.

8. The method according to claim 1, further comprising determining whether the pre-treatment algorithm is complete before applying the energy to the pre-treated, grasped tissue in accordance with the tissue sealing algorithm.

9. The method according to claim 8, wherein determining whether the pre-treatment algorithm is complete includes comparing an overall impedance change to an impedance change threshold.

10. The method according to claim 1, further comprising, after applying the energy to the grasped tissue in accordance with the pre-treatment algorithm and before applying the energy to the pre-treated, grasped tissue in accordance with the tissue sealing algorithm, implementing a delay period where no energy is applied.

11. A method of sealing tissue, comprising:
grasping tissue between first and second jaw members;
determining whether pre-treatment of the grasped tissue is to be performed;
in a case where it is determined that pre-treatment of the grasped tissue is to be performed:
applying energy to the grasped tissue in accordance with a pre-treatment algorithm to pre-treat the grasped tissue in anticipation of tissue sealing; and
applying energy to the pre-treated, grasped tissue in accordance with a tissue sealing algorithm to seal the pre-treated grasped tissue; and
in a case where it is not determined that pre-treatment of the grasped tissue is to be performed:
applying energy to the grasped tissue in accordance with the tissue sealing algorithm to seal the grasped tissue.

12. The method according to claim 11, wherein the tissue sealing algorithm is independent of the pre-treatment algorithm.

13. The method according to claim 11, wherein determining whether pre-treatment of the grasped tissue is to be performed is based on sensor feedback.

14. The method according to claim 11, wherein determining whether pre-treatment of the grasped tissue is to be performed is based on a determined size of the grasped tissue.

15. The method according to claim 11, wherein determining whether pre-treatment of the grasped tissue is to be performed is based on a manual input.

16. The method according to claim 11, wherein determining whether pre-treatment of the grasped tissue is to be performed is based on at least one of: a clamping force applied by the first and second jaw members to the grasped tissue or a gap distance between opposing surfaces of the first and second jaw members with the grasped tissue therebetween.

17. The method according to claim 11, further comprising, in the case where it is determined that pre-treatment of the grasped tissue is to be performed, determining whether the pre-treatment algorithm is complete before applying the energy to the pre-treated, grasped tissue in accordance with the tissue sealing algorithm.

18. The method according to claim 11, wherein determining whether the pre-treatment algorithm is complete includes determining an overall impedance change during the pre-treatment.

19. The method according to claim 11, wherein determining whether the pre-treatment algorithm is complete includes at least one of: determining a size of the grasped tissue during the pre-treatment; determining a clamping force applied by the first and second jaw members to the grasped tissue during the pre-treatment; or determining a gap distance between opposing surfaces of the first and second jaw members during the pre-treatment.

20. The method according to claim 11, wherein:
at least a portion of the pre-treatment algorithm is voltage-controlled; and
at least a portion of the tissue sealing algorithm adjusts energy output to track an impedance versus time trajectory.
